(19) United States
(12) Patent Application Publication
(10) Pub. No.: US 2026/0000694 A1
(43) Pub. Date: Jan. 1, 2026

(54) METHODS AND COMPOSITIONS RELATED TO PREBIOTIC FORMULATIONS USEFUL IN PROMOTING UROGENITAL HEALTH

(71) Applicant: KIMBERLY-CLARK WORLDWIDE, INC., Neenah, WI (US)

(72) Inventors: Jane BALLESTEROS, Oshkosh, WI (US); Kathleen C. ENGELBRECHT, Kaukauna, WI (US)

(21) Appl. No.: 18/997,917

(22) PCT Filed: Jul. 27, 2023
(86) PCT No.: PCT/US2023/071110

§ 371 Date: Jan. 23, 2025

Related U.S. Application Data
(60) Provisional application No. 63/393,341, filed on Jul. 29, 2022.

(51) Int. Cl. A61K 31/7016; A61K 31/702; A61P 31/04; A61P 43/00

(52) U.S. Cl. CPC A61K 31/7016; A61K 31/702; A61P 31/04; A61P 43/00

(57) ABSTRACT

Prebiotic formulations can modulate the growth and/or activity of microorganisms in the urogenital area. Such prebiotic formulations can comprise isomaltulose along with at least one other prebiotic, such as 1-kestose or lactulose. These microorganisms which are modulated can be directly related to urogenital health, and their modulation can be used for the prevention, control, or treatment of dysbiosis of the urogenital area.

### CROSS-REFERENCE TO RELATED APPLICATIONS

This application claims benefit of U.S. Provisional Application No. 63/393,341, filed Jul. 29, 2022, incorporated herein by reference in its entirety.

### BACKGROUND

Prebiotics are a group of nutrients that are degraded by beneficial microorganisms found in the human body. The relationship of these microorganisms with overall health has been an area of increasing interest in recent years. Prebiotics are most commonly plant fibers selectively used by healthy bacteria as a food source to support their growth.

The term “microbiota” refers to the group of microorganisms that inhabit a specific biological niche. Many of these specific niches exist on the human body, such as, for example, in the gut, the skin, the nasopharynx, the mouth, and the urogenital region. The vaginal and urinary microbiota represent important microbial niches. Urogenital microbiota can form a mutually beneficial relationship with their host and have major impact on health and disease. Lactobacillus make up the majority of bacteria in the vagina and include Lactobacillus crispatus (“L. crispatus”), Lactobacillus gasseri, Lactobacillus iners, and Lactobacillus jensenii (Verhelst et al. 2005; Chee et al. 2020). Keeping a proper balance between bacteria species and ensuring that beneficial bacteria (e.g., L. crispatus) are thriving is critical to urogenital health.

To maintain proper urogenital health, it is important to both encourage the growth of beneficial microbes and reduce the growth of unwanted microbes. Uropathogenic Escherichia coli (UPEC), Proteus mirabilis, Klebsiella pneumoniae, Staphylococcus saprophyticus, Enterococcus faecalis, and Streptococcus anginosus (“S. anginosus”) are some of the bacteria responsible for imbalances and infections in the urogenital region. Controlling populations of unwanted microbes can help control or prevent infections, or mitigate symptoms, of urinary tract infections (UTI), urgency urinary incontinence (UUI), and overactive bladder (OAB), for example.

Isomaltulose, also referred to herein by the trade name Palatinose™, is a prebiotic which naturally occurs in such products as honey and sugar cane juice. Isomaltulose has been shown to promote the proliferation of select probiotics (Su et al. 2021). Furthermore, the use of isomaltulose as a prebiotic in vaginal applications has been discussed in the art (U.S. Pub. No. 2016/0375045 A1 and U.S. Pub. No. 2019/0231748 A1). However, there is an on-going need in the art for prebiotic formulations that offer enhanced support for urogenital health by encouraging the growth of beneficial microbes (e.g., Lactobacillus) while reducing the growth of unwanted microbes (e.g., S. anginosus).

### SUMMARY

Disclosed herein is a prebiotic formulation comprising prebiotic agents isomaltulose and at least one of 1-kestose and lactulose. The combination of isomaltulose and at least one additional prebiotic (1-kestose or lactulose) results in an unexpected and synergistic reaction that is capable of increasing the growth or activity of beneficial microorganisms (e.g., L. crispatus) and decreasing the growth of activity of harmful or unwanted bacteria (e.g., S. anginosus).

Also disclosed is a method of modulating growth of urogenital microorganisms in a subject, the method comprising administering to the subject a prebiotic formulation, wherein the prebiotic formulation comprises prebiotic agents, and wherein the prebiotic agents comprise isomaltulose and at least one of 1-kestose and lactulose.

Further disclosed is a method of treating or preventing urogenital dysbiosis, the method comprising administering to the subject a prebiotic formulation, wherein the prebiotic formulation comprises prebiotic agents, and wherein the prebiotic agents comprise isomaltulose and at least one of 1-kestose and lactulose.

Additional aspects and advantages of the disclosure will be set forth, in part, in the detailed description and any claims which follow, and in part will be derived from the detailed description or can be learned by practice of the various aspects of the disclosure. The advantages described below will be realized and attained by means of the elements and combinations particularly pointed out in the appended claims. It is to be understood that both the foregoing general description and the following detailed description describe examples, are explanatory, and are not restrictive of the disclosure.

### BRIEF DESCRIPTION OF THE DRAWINGS

The accompanying drawings, which are incorporated in and constitute a part of this specification, illustrate certain examples of the present disclosure and, together with the description, serve to explain, without limitation, the principles of the disclosure. Like numbers represent the same elements throughout the figures.

FIG. 1 is a chart showing the average log CFU/mL change (Δ) in bacterial recovery for Lactobacillus crispatus KC18-1173-1 and Streptococcus anginosus KC18-1131-3B after exposure to prebiotics in a competition assay. Error bars indicate the standard error of the mean. Statistically significant differences are indicated by (*) (p<0.05).

FIG. 2 is a chart showing the average log CFU/mL change (4) in bacterial recovery for Lactobacillus crispatus KC18-1173-1 and Streptococcus anginosus KC18-1131-3B after exposure to various combinations of isomaltulose and 1-kestose or isomaltulose and lactulose in a competition assay. Error bars indicate the standard error of the mean. Statistically significant differences are indicated by (*) (p<0.05).

### DETAILED DESCRIPTION

### Definitions

As used herein, the singular forms “a,” “an,” and “the” include plural referents unless the context clearly dictates otherwise. Thus, for example, “a compound” includes mixtures of compounds.

As used herein, the term “about” or “approximately” means within an acceptable error range for the particular value as determined by one of ordinary skill in the art, which will depend in part on how the value is measured or determined (i.e., the limitations of the measurement system). For example, “about” can mean within 3 or more than 3 standard deviations, per the practice in the art. Alternatively, “about” can mean a range of up to 20%, or up to 10%, or up to 5%, or up to 1% of a given value. Alternatively, particularly with respect to biological systems or processes, the term can mean within an order of magnitude, or within 5-fold, or within 2-fold, of a value.

As used herein, the terms “inhibit,” “inhibiting,” and “inhibition” mean to reduce by a measurable amount or to prevent entirely (e.g., by a decrease in activity, response, condition, disease, or another biological parameter).

As used herein, “w/v” (or “wt/vol %” or “wt/vol”) refers to the value obtained by dividing the weight of a substance (in grams) by the volume of the solution (in milliliters), and then multiplying by 100.

As used herein, “urogenital” refers to the vulva, vagina, urinary tract, bladder, and surrounding areas.

As used herein, the term “therapeutic effect” refers to the ability of the compositions and formulations of the present disclosure to stimulate the growth of beneficial microbes (e.g., L. crispatus) relative to unwanted microbes (e.g., S. anginosus) measured according to the therapeutic effect protocol described below. The therapeutic effect can be expressed as a ratio of beneficial microbes to unwanted microbes.

As used herein, a “prebiotic” refers to an ingredient that allows specific changes, both in the composition and/or activity in the urogenital microbiota that may (or may not) confer benefits upon a host subject. In some instances, the prebiotic can be added to a material which is worn by the host subject, such as a diaper, pad, or liner, for example. In other instances, the prebiotic can be applied to the urogenital area in the form of a cream, gel, spray, or suppository, for example. The prebiotic can also be applied in the form of a wipe. In addition to the combination of prebiotics disclosed herein, the prebiotic can include additional complex carbohydrates, amino acids, peptides, minerals, or other essential nutritional components for the survival of the bacterial composition.

The “colonization” of a host organism includes the non-transitory residence of a bacterium or other microscopic organism. As used herein, “reducing colonization” of a host subject's urogenital tract (or any other microbiota niche) by a pathogenic or non-pathogenic bacterium includes a reduction in the residence time of the bacterium in the urogenital tract as well as a reduction in the number (or concentration) of the bacterium in the urogenital tract. The reduction in colonization can be permanent or occur during a transient period of time.

A “combination” of two or more bacteria includes the physical co-existence of the two bacteria, either in the same material or product or in physically connected products, as well as the temporal co-administration or co-localization of the two bacteria.

As used herein, “treatment,” “treat,” or “treating” means a method of reducing the effects of a disease or condition. Treatment can also refer to a method of reducing the disease or condition itself rather than just the symptoms. The treatment can be any reduction from pre-treatment levels and can be but is not limited to the complete ablation of the disease, condition, or the symptoms of the disease or condition.

As used herein, “dysbiosis” refers to a state of the microbiota or microbiome of the urogenital tract (or any other microbiota niche) in which the normal diversity and/or function of the ecological network is disrupted. Any disruption from the preferred (e.g., ideal) state of the microbiota can be considered a dysbiosis, even if such dysbiosis does not result in a detectable decrease in health. This state of dysbiosis may be unhealthy (e.g., result in a diseased state), or it may be unhealthy under only certain conditions, or it may prevent a subject from becoming healthier. Dysbiosis may be due to a decrease in diversity of the microbiota population composition, the overgrowth of one or more population of pathogens (e.g., a population of pathogenic bacteria) or pathobionts, the presence of and/or overgrowth of symbiotic organisms able to cause disease only when certain genetic and/or environmental conditions are present in a patient, or the shift to an ecological network that no longer provides a beneficial function to the host and therefore no longer promotes health.

As used herein, “microbiota” refers to the community of microorganisms that inhabit (sustainably or transiently) a specific biological niche, such as a distinct region of the host subject, (e.g, a mammal such as a human), including, but not limited to, eukaryotes (e.g., protozoa), archaea, bacteria, and viruses (including bacterial viruses, i.e., a phage).

As used herein, “microbiome” refers to the genetic content of the communities of microbes that live in and on specific niches of the host subject, both sustainably and transiently, including eukaryotes, archaea, bacteria, and viruses (including bacterial viruses (i.e., phage)), wherein “genetic content” includes genomic DNA, RNA such as ribosomal RNA, the epigenome, plasmids, and all other types of genetic information.

As used herein, “ecological niche” or simply “niche” refers to the ecological space that an organism or group of organisms (e.g., a bacterial population) occupies. Niche describes how an organism (or population of organisms) responds to the distribution of resources, physical parameters (e.g., host tissue space), and competitors (e.g., by growing when resources are abundant and/or when predators, parasites, and pathogens are scarce) and how it in turn alters those same factors (e.g., by limiting access to resources by other organisms or acting as a food source for predators).

As used herein “preventing” or “prevention” refers to any methodology where the disease state does not occur due to the actions of the methodology (such as, for example, administration of a probiotic and/or a prebiotic as described herein). In one aspect, it is understood that prevention can also mean that the disease is not established to the extent that occurs in untreated controls. For example, there can be a 5, 10, 15, 20, 25, 30, 35, 40, 50, 60, 70, 80, 90, or 100% reduction in the establishment of disease frequency relative to untreated controls. Accordingly, prevention of a disease encompasses a reduction in the likelihood that a subject will develop the disease relative to an untreated subject (e.g., a subject who does not receive a prebiotic as described herein).

As used herein, the term “subject” or “host subject” refers to any organism or animal subject that is an object of a method or material (e.g., humans). Synonyms used herein include “patient” and “host.” In some implementations, the subject or host may be suffering from a dysbiosis of the urogenital area. Examples include, but are not limited to, urinary tract infections (UTIs) and urgency urinary incontinence (UUI), a common form of urinary incontinence (UI), and overactive bladder (OAB).

As used herein, “synergy” or “synergistic” refers to the interaction or cooperation of two or more entities to produce a combined effect greater than the sum of their separate effects. For example, “synergy” between two or more prebiotics can result in the inhibition of one or more pathogen's ability to grow or can result in an increase of the ability of a beneficial microbe to grow.

### General Description

Disclosed herein are prebiotic formulations which can modulate the growth and/or activity of microorganisms in the urogenital area. These modulated microorganisms can be directly related to urogenital health, and their modulation can be used for the prevention, control, or treatment of dysbiosis of the urogenital area. The female urogenital system has two major subdivisions, the urinary tract and the genital tract. The urinary tract is comprised of the kidneys, ureters, urinary bladder, and urethra. The genital tract consists of the clitoris, vagina, ovaries, and uterus.

The prebiotic formulation disclosed herein comprises isomaltulose along with at least one other prebiotic selected from 1-kestose and lactulose. It is important to note that the combination of isomaltulose and at least one additional prebiotic (1-kestose or lactulose) results in an unexpected synergistic reaction which is capable of increasing the growth or activity of beneficial microorganisms and decreasing the growth of activity of harmful or unwanted bacteria.

The urogenital microbiota that can be modulated by the prebiotic formulations disclosed herein include, but are not limited to, those of the genera Lactobacillus and Lactococcus. Lactobacilli and lactococci are lactic acid bacteria that are known for their ability to produce lactic acid as a sole or primary end product of carbohydrate metabolism. They can prevent the adherence, growth, and colonization of uropathogenic bacteria. Accordingly, increasing the population or activity of these microorganisms can be beneficial to the host, and can be used to treat or prevent dysbiosis, which can lead to certain infections, diseases, or disorders. For example, it has been shown that healthy microbial populations of lactobacilli have a strong inhibitory effect on the potentially pathogenic bacteria Escherichia. coli (Akgül et al. 2018). Examples of beneficial lactic acid producing bacteria (lactobacilli and lactococci) of the urogenital tract include, but are not limited to, Lactobacillus crispatus, Lactobacillus iners, Lactobacillus jensenii, Lactobacillus gasseri, Lactobacillus paragasseri, Lactobacillus johnsonii, Lactobacillus mucosae, Lactobacillus mulieris, Lactobacillus fermentum, Lactobacillus plantarum, Lactobacillus brevis, Lactobacillus casei, Lactobacillus paracasei, Lactobacillus delbrueckii, Lactobacillus helveticus, Lactobacillus acidophilus, Lactobacillus rhamnosus, Lactobacillus reuteri, and Lactococcus lactis. In a particular implementation, L. crispatus growth can be increased in the presence of the prebiotic formulations described herein.

By “increase in population of beneficial bacteria” is meant that the population is increased by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% as compared to a population which has not been exposed to the prebiotic formulation, or which has been exposed to only one component of the prebiotic formulation, but not to both isomaltulose and at least one additional prebiotic (1-kestose or lactulose).

The prebiotic formulations disclosed herein can also be used to reduce the growth or activity of unwanted, detrimental, or harmful microorganisms in the urogenital area. Examples of such bacteria include Escherichia coli, Proteus mirabilis, Klebsiella pneumoniae, Staphylococcus saprophyticus, Enterococcus faecalis, Streptococcus anginosus, Candida albicans, Bacteroides fragilis, Peptostreptococcus anaerobius, Prevotella bivia, Atopobium vaginae, Mobiluncus mulieris, and Gardnerella vaginalis.

By “decrease in population of harmful or unwanted bacteria” is meant that the population is decreased by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% as compared to a population which has not been exposed to the prebiotic formulation, or which has been exposed to only one component of the prebiotic formulation, but not to both isomaltulose and at least one additional prebiotic (1-kestose or lactulose).

Inhibition of a pathogen or non-pathogen encompasses the inhibition of any desired function or activity of the pathogen or non-pathogen. Demonstrations of inhibition, such as a decrease in the growth of a pathogenic bacterial cell population or a reduction in the level of colonization of a pathogenic bacterial species, are provided herein and otherwise recognized by one of ordinary skill in the art. Inhibition of a pathogenic or non-pathogenic bacterial population's “growth” may include inhibiting an increase in the size of a pathogenic or non-pathogenic bacterial cell population and/or inhibiting the proliferation (or multiplication) of a pathogenic or non-pathogenic bacterial cell population. Inhibition of colonization of a pathogenic or non-pathogenic bacterial species may be demonstrated by measuring and comparing the amount or burden of the bacterial species before and after a treatment. As used herein, inhibition includes cytostatic and/or cytotoxic activities.

### Prebiotic Compositions

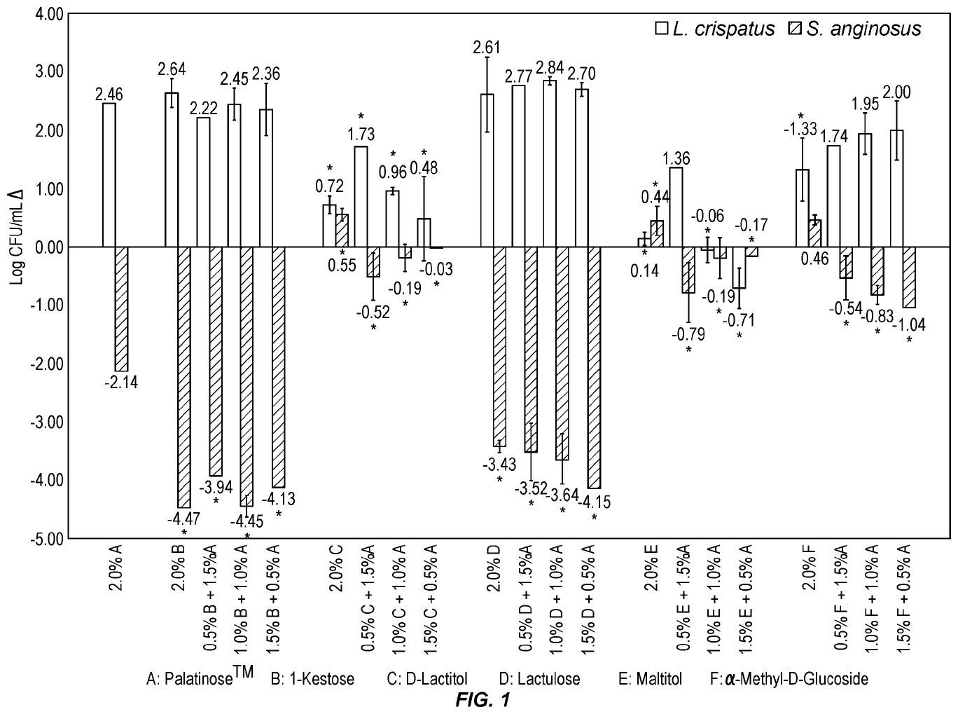

The prebiotic formulations disclosed herein comprise, as a first component, isomaltulose or a derivative thereof. Isomaltulose is sold under the tradename Palatinose™. Isomaltulose (6-O-α-D-glucopyranosyl-D-fructose) is a disaccharide carbohydrate composed of glucose and fructose. The glucose and fructose are linked by an alpha-1,6-glycosidic bond. Derivatives of isomaltulose are known to those of skill in the art and herein contemplated (Weysser 2022; Barea-Alvarez 2014; Kashimura 2008, herein incorporated by reference in their entirety for their teachings concerning derivatives of isomaltulose). Examples of derivatives include isomaltooligosaccharides, which are glucose oligomers with α-D-(1,6)-linkages (examples are isomaltose, panose, isomaltotriose, isomaltotetraose, and isomaltopentaose). These isomaltooligosaccharides can comprise 3, 4, 5, 6, 7, 8, or 9 monosaccharide units. While it has previously been found that isomaltulose is useful in modulating the microbiota of the urogenital area (U.S. Patent Application Publication Nos. 2016/0375045 A1 and 2019/0231748 A1, herein incorporated by reference in their entirety for their teaching concerning the use of isomaltulose to treat urogenital disorders), it has now been surprisingly and unexpectedly discovered that isomaltulose in combination with 1-kestose or lactulose, or derivatives thereof, provides synergistic results in modulating the urogenital microbiota. The synergistic combination of these prebiotics is described in Example 1 and can be seen in FIG. 1.

1-Kestose is a trisaccharide found in vegetables consisting of beta-D-fructofuranose having beta-D-fructofuranosyl and alpha-D-glucopyranosyl residues attached at the 1- and 2-positions respectively. Methods of obtaining 1-kestose are known to those of skill in the art (US20150232898A1, herein incorporated by reference in its entirety for its teaching concerning obtaining 1-Kestose). Lactulose is a synthetic galactosylfructose disaccharide (available through Biosynth®, for example). Also disclosed are derivatives of lactulose.

By “at least one of” 1-kestose and lactulose, it is meant that at least one of these compounds (1-kestose or lactulose) is present along with isomaltulose. So, for example, the prebiotic formulation can comprise (i) isomaltulose and 1-kestose or (ii) isomaltulose and lactulose.

The prebiotic formulation disclosed herein can comprise a carrier. The carrier can be physiologically compatible with the dermal or epithelial tissue of the subject to which it is administered. For example, the carrier can be a “dermatologically acceptable carrier,” which refers to a carrier that is suitable for topical application to the urogenital area. That is, the carrier is preferably substantially inactive except for surfactant properties used in making a suspension of the active ingredients. The compositions may include other physiologically active constituents that do not interfere with the efficacy of the active agents in the composition.

The dermatologically acceptable carrier may be in a wide variety of forms such as, for example, simple solutions (water-based or oil-based), solid forms (e.g., gels or sticks) and emulsions. In some implementations, the carrier can be either aqueous or non-aqueous. Water is a particularly preferred aqueous carrier. Non-aqueous carriers may include, for example, glycols, such as propylene glycol, butylene glycol, triethylene glycol, hexylene glycol, polyethylene glycols, ethoxydiglycol, and dipropyleneglycol; alcohols, such as ethanol, n-propanol, and isopropanol; triglycerides; ethyl acetate; acetone; triacetin; and combinations thereof.

The carrier can constitute greater than 75% w/v of the prebiotic formulation. For example, the carrier can constitute 75, 76, 77, 78, 79, 80, 81, 82, 83, 84, 85, 86, 87, 88, 89, 90, 91, 92, 93, 94, 95, 96, 97, 98, or 99% w/v of the pharmaceutical composition, or any amount above, below, or in-between these values.

The prebiotic compounds are considered the “active portion” of the prebiotic formulation. The total weight of the prebiotic compounds (as a whole, meaning isomaltulose as well as at least one further prebiotic compound) in the prebiotic formulation can be 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9, or 25.0%, or any amount below, above, or in-between these values. In a preferred implementation, the total prebiotic compounds comprise 0.5 to 10% w/v. In another preferred implementation, the prebiotic compounds comprise 0.5 to 5% w/v. In yet another preferred implementations, the prebiotic compounds comprise 2% w/v.

Specifically, isomaltulose can be present in the prebiotic formulation at an amount of 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, or 12.5% w/v, or any amount above, below, or in between these values.

The additional prebiotic compound of the formulations (1-kestose or lactulose, or derivatives thereof) can be present at an amount of 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, or 12.5% w/v, or any amount above, below, or in between these values.

By way of example, when the prebiotic formulation comprises isomaltulose and one additional prebiotic compound selected from the group comprising 1-kestose and lactulose, the isomaltulose can be present at 0.5% w/v and the additional prebiotic can be present at 1.5% w/v. In another implementation, the isomaltulose can be present at 0.7% w/v and the additional prebiotic compound can be present at 1.3% w/v. Alternatively, the isomaltulose can be present at 0.8% w/v and the additional prebiotic compound can be present at 1.2% w/v. In another implementation, the isomaltulose can be present at 1.0% w/v and the additional prebiotic compound can be present at 1.0% w/v. Alternatively, the isomaltulose can be present at 1.2% w/v and the additional prebiotic compound can be present at 0.8% w/v. Alternatively, the isomaltulose can be present at 1.3% w/v and the additional prebiotic compound can be present at 0.7% w/v. Alternatively, the isomaltulose can be present at 1.5% w/v and the additional prebiotic compound can be present at 0.5% w/v. In another implementation, the isomaltulose can be present at 2.0% w/v and the additional prebiotic compound can be present at 1.0% w/v. Alternatively, the isomaltulose can be present at 2.0% w/v and the additional prebiotic compound can be present at 2.0% w/v. In another implementation, the isomaltulose can be present at 2.0% w/v and the additional prebiotic compound can be present at 0.2% w/v. Alternatively, the isomaltulose can be present at 2.0% w/v and the additional prebiotic compound can be present at 0.1% w/v. In another implementation, when isomaltulose is present at 1.5% w/v, the additional prebiotic can be present at 1.5% w/v. Put another way, the ratio of isomaltulose to the additional prebiotic compound can be 20:1, 13:7, 10:1, 5:1, 4:1, 3:1, 3:2, 2:1, 1:1, 1:2, 2:3, 1:3, 1:4, 1:5, 7:13, or any amount above, below, or in-between these ratios.

The prebiotic formulation can also comprise probiotic compositions. This can include live, active cultures. Examples of such cultures are known to those of skill in the art and can be found in U.S. patent application No. 20200138881A1, Japanese Patent Application JP6509822B2, PCT Application No. WO2022100633A1, and Chinese Patent Application CN110960561A, all of which are incorporated in their entirety for their teachings concerning probiotic compositions which can be topically administered to the urogenital region. Specific examples include L. crispatus (Li et al. 2019, herein incorporated by reference in its entirety for its disclosure concerning topical administration of L. crispatus), Lactobacillus gasseri, Lactobacillus fermentum, and Lactobacillus rhamnosus.

The prebiotic formulation disclosed herein can include known antimicrobial agents, known antiviral agents, known antifungal agents, all of which must be compatible with maintaining viability of the prebiotic compounds. The other agents in the compositions can be either synergists or active agents. The compositions may also include known antioxidants, buffering agents, and other agents to enhance appearance, smell, or texture. Thickening agents may be added to the compositions such as polyvinylpyrrolidone, polyethylene glycol or carboxymethylcellulose.

Other suitable additives that may be included in the compositions of the present disclosure include compatible colorants, deodorants, emulsifiers, anti-foaming agents (when foam is not desired), lubricants, skin conditioning agents, skin protectants and skin benefit agents (e.g., aloe vera and tocopheryl acetate), solvents (e.g., water soluble glycol and glycol ethers, glycerin, water soluble polyethylene glycols, water soluble polyethylene glycol ethers, water soluble polypropylene glycols, water soluble polypropylene glycol ethers, dimethylisosorbide), solubilizing agents, suspending agents, builders, (e.g., alkali and alkaline earth metal salts of carbonate, bicarbonate, phosphate, hydrogen phosphate, dihydrogen phosphate, sulfate hydrogen sulfate), wetting agents, pH adjusting ingredients (a suitable pH range of the compositions can be from 3.5 to 8), chelators, propellants, dyes and/or pigments, and combinations thereof.

### Methods of Using Prebiotic Formulations

Disclosed herein are methods of modulating the growth of urogenital microorganisms in a subject, the method comprising administering to the subject a prebiotic formulation, wherein the prebiotic formulation comprises prebiotic agents, and wherein the prebiotic agents comprise isomaltulose and at least one of 1-kestose and lactulose. As described above, modulation can mean an increase in growth of desirable microorganism, and/or a decrease, or reduction, in growth of an undesired microorganism.

Specifically, disclosed herein is a method of treating or preventing urogenital dysbiosis, the method comprising administering to the subject a prebiotic formulation, wherein the prebiotic formulation comprises prebiotic agents, and wherein the prebiotic agents comprise isomaltulose and at least one of 1-kestose and lactulose, as described above.

Dysbiosis can lead to a variety of diseases, disorders, infections, and other uncomfortable or unwanted physical effects. Examples include, but are not limited to, overactive bladder, urinary urge incontinence, or urinary tract infection.

Before administration of the prebiotic composition, the subject may have been diagnosed with having an overactive bladder, urinary urge incontinence, or urinary tract infection. For example, a physician or other caregiver can provide such a diagnosis. In another example, the subject may have been diagnosed as being susceptible to an overactive bladder, urinary urge incontinence, or urinary tract infection. In another example, the subject may use the prebiotic composition as a prophylactic, or means of preventing an overactive bladder, urinary urge incontinence, or urinary tract infection. The methods and compositions disclosed herein can be used as a routine part of health or can be used as needed.

The prebiotic formulation disclosed herein can be administered topically. For example, the prebiotic formulation can be placed on, or integrated into, an absorbent product which comes into contact with the urogenital region of the subject, such as a sanitary product (sanitary pad or tampon), a diaper, a tissue, toilet paper, or a wipe. Further examples include a dermal patch, adhesive tape, clothing, panty protector, or incontinence guard. The prebiotic formulation can be placed onto or into such a product before it reaches the end user, such that the formulation is integrated into the product before it reaches the subject, or it can be placed onto the product immediately before use by the subject. When this is the case, the formulation can be placed on the substrate in the form of a spray, lotion, cream, soap, liquid, or gel, for example. In either case, the use of the absorbent product coated with the prebiotic formulation provides contact between the urogenital area and the absorbent product, and thereby contacts the target area with an effective amount of the prebiotic formulation.

The compositions of the present disclosure may be applied to a suitable substrate, which in-turn may be used to apply the therapeutic agent to a user. Suitable applicators include a web, such as a wet laid tissue web or air laid web, gauze, cotton swab, transdermal patch, container, or holder. Particularly preferred applicators include fibrous webs, including flushable and non-flushable cellulosic webs and nonwoven webs of synthetic fibrous material. Useful webs may be wet laid, air laid, meltblown, or spunbonded. Suitable synthetic fibrous material includes meltblown polyethylene, polypropylene, copolymers of polyethylene and polypropylene, bicomponent fibers including polyethylene or polypropylene, and the like. Useful nonwoven webs may be meltblown, coform, spunbond, airlaid, hydroentangled nonwovens, spunlace, or bonded carded webs.

In certain implementations, particularly those in which the composition is applied to a web, it may be desirable that the formulation provide certain physical attributes, such as having a smooth, lubricious, non-greasy feel; the ability to at least partially transfer from the web to the user; the capability to be retained on the web at room temperature; or the ability to be compatible with the web manufacturing process. In certain implementations, it is preferred that at least a portion of the composition is transferred from the tissue to the user in use.

The composition may be applied to a web during formation of the web or after the web has been formed and dried, often referred to as off-line or post-treatment. Suitable methods of applying the composition to a web include methods known in the art such as gravure printing, flexographic printing, spraying, WEKO™, slot die coating, or electrostatic spraying. One particularly preferred method of offline application is rotogravure printing.

### Articles of Manufacture

Also disclosed herein are various articles of manufacture which combine the prebiotic formulation described herein with various medical or personal hygiene devices so as to modulate the urogenital microbiota. Therefore, contemplated herein is a prebiotic formulation comprising isomaltulose, along with one other prebiotic, such as 1-kestose or lactulose, applied to a solid surface or impregnated into a solid matrix of any device or article of manufacture that is intended to be in contact with the urogenital area of the subject. Preferably the solid surface is a flexible substrate than can be worn on or wiped on the skin or mucous membrane. The solid surface can be absorbent, for example.

Many different types of absorbent products having absorbent structures are well known in the art, and can include diapers, towelettes (e.g., baby wipes or feminine hygiene towelettes), sanitary napkins, tampons, panty protectors, dermal patches, adhesive tape, bandages, wound or sore dressings, absorbent pads, incontinence guards, bed sheets or protectors, saliva absorbent, articles of clothing (e.g., underclothes, sleeping apparel), bath towels, wash cloths, toilet paper, tissues, and the like. Thus, described herein is an absorbent product comprising an aqueous liquid absorbent structure and an effective amount of the prebiotic formulation described herein.

Absorbent structures in sanitary products (e.g., absorbent products, such as Poise® products) are typically produced by fluffing cellulosic or other fibrous pulp into a roll, bale, or sheet, for instance, to form a pulp mat, sometimes admixed with so-called superabsorbent materials in the pulp mat. The superabsorbent materials are typically polymeric formulations capable of absorbing many times their own weight of water of body fluid, and are well known in the art. The pulp mat is typically compressed so as to enhance its fluid-wicking ability and also in order to reduce pulp body bulk, and therewith obtain an article which is as compact as possible to achieve the absorbent properties desired in the particular sanitary product.

The absorbent structure may also include other constituents, for example, components which will improve fluid acquisition properties, fluid-wicking properties, fluid retention properties, and the like well-known in the art. Other included constituents include components which increase coherent strength (i.e., the ability to withstand deformation during use). The absorbent structure may contain fibrous woven, knitted or non-woven materials, occlusive or non-occlusive films or membranes, granules, pellets or aggregates of absorbent material, synthetic polymer fibers, films, membranes and foams (e.g., nylon, polytetrafluoroethylene (PTFE, such as Teflon® or Gor-Tex®), polystyrene, polycarbonate, polyvinylchloride and polysulphone). All of these forms are well known in the art and include, for example, knitted or woven fabrics, non-woven fabrics such as felt and batting, fiber balls of cotton, rayon, cellulose or synthetic fibers, and like materials.

The fibers can be natural fibers, including but not limited to wool, silk, cotton, cellulosic fiber, and the like natural fibers. Natural polymers based on polysaccharide can also be used, including, but not limited to: modified cellulose and cellulose derivatives (e.g., alkyl-, hydroxyalkyl-, carboxymethylcellulose); gum resins (e.g., guar gum, locust bean gum, tragacanth gum, gum arabic, pectin, etc.); starch and starch derivatives (e.g., corn starch, grain starch, potato starch, amylose, amylopectin, dextrin, dextran, modified starch, hydroxy-ethyl starch, cationic starch, starch graft polymers, and the like polymers). The fibers can be synthetic fibers, including, but not limited to: polyester, polyolefin, polyamide, polyvinyl alcohol, polyvinyl acetate, polyvinyl chloride, polyvinyl urea, polyurethane, polyurea, polyacrylonitrile, as well as copolymers of these polymers, and the like synthetic fibers.

The absorbent product can be formatted into a multi-layer configuration, having an absorbent structure layer, a fluid permeable top layer which allows wicking of fluid but is itself non-wettable due to its structural composition (e.g., synthetic fiber construction), and a fluid-impermeable bottom layer (i.e., back sheet) which prevents absorbed fluid to pass from the absorbent structure layer to the adjacent tissues of the user when contacted by the absorbent product during use. Such layered configurations are well known in the diaper and panty liner arts.

The prebiotic formulation can be applied to the solid surface using any of a variety of known methods including, for example, applying a powder, spray drying the prebiotic formulation onto the material or soaking the material in a solution containing the prebiotic formulation and then using the wetted material or drying the material before use. Porous material may contain the prebiotic compounds in the pores or interstices of the solid material. The prebiotic formulation can be attached by adhesion, such as by attachment to an adhesive layer that is then applied to the skin (e.g., in a bandage or dermal patch). The prebiotic formulation and/or the isolated active agent can be impregnated into the solid material during the manufacturing process of the flexible article (e.g., added to a synthetic composition before or during the polymerization process).

Any of the solid materials comprising the prebiotic formulation can be packaged individually or in groups, suitable for holding the treated material using standard packaging materials (e.g., in a shrink wrapper, sealed packet, protective wrapper or dispensing container suitable for holding dry or wet materials) The article of manufacture can have applied thereon any of the additional/optional components of a composition of this disclosure, including carriers, disinfectants, antibacterial agents, salts, FOS, and the like. In particular, the absorbent product can include as a component part of the absorbent structure inert ingredients, neutral filling agents, and the like. Examples of neutral filling agents include peat, sand, clay, garden mold, ground shells of nuts or pomaceous fruit, wood flour, chitin-containing flour, and the like well-known materials.

Unless defined otherwise, all scientific and technical terms used herein have the same meaning as commonly understood by those skilled in the relevant art. Unless mentioned otherwise, the techniques employed or contemplated herein are standard methodologies well known to one of ordinary skill in the art. The Examples are for illustration only.

### EXAMPLES

To further illustrate the principles of the present disclosure, the following examples are put forth to provide those of ordinary skill in the art with a complete disclosure and description of how the compositions, articles, and methods claimed herein are made and evaluated. They are intended to be examples and are not intended to limit the scope of what the inventors regard as their disclosure. Efforts have been made to ensure accuracy with respect to numbers (e.g., amounts, temperatures, etc.); however, some errors and deviations should be accounted for. Unless indicated otherwise, temperature is ° C. or is at ambient temperature, and pressure is at or near atmospheric. There are numerous variations and combinations of process conditions that can be used to optimize product quality and performance. Only reasonable and routine experimentation will be required to optimize such process conditions.

### Example 1: Screening Prebiotic Combinations in a Co-Culture Assay

The prebiotic Palatinose™/isomaltulose confers a selective advantage to clinical strains of Lactobacillus crispatus. In this example, the prebiotic was combined with one of five additional compounds (1-kestose, D-lactitol, lactulose, maltitol, and α-Methyl-D-Glucoside). This was done to assess if the effects of Palatinose™ (i.e., the increased recovery of L. crispatus and decreased recovery of S. anginosus in a co-culture system) could be synergized or further increased when combined with a secondary compound. The prebiotic compounds mixed with Palatinose™/isomaltulose are listed in Table 1. The five additional compounds were selected based on their positive or neutral L. crispatus recovery in competition assays with the individual compounds when tested against S. anginosus. The compound mixtures are listed in Table 2.

### Methods

The methods used herein were adapted from Vongsa et al. The method used in this particular example is as follows:
1. A stock was created using a single prebiotic, a prebiotic mixture, or the positive control (glucose) in API 50 CHL media (Biomerieux, Marcy-l'Étoile, France). API 50 CHL media containing minimal amounts of carbon for growth and bromocresol purple as a pH indicator of fermentation. The stock media was filter-sterilized with a 0.22 μM filter and store at 4-6° C.
2. From a freezer stock, the bacteria were subcultured twice into De Man, Rogosa, and Sharpe (MRS) broth (BD Difco, Becton Dickinson, Franklin Lakes, NJ) and incubated at 37° C. anaerobically overnight until stationary.
3. The second MRS broth subculture was plated onto an MRS agar plate for Lactobacillus crispatus KC18-1173-1 or blood agar for Streptococcus anginosus KC18-1131-3B and incubated at 37° C. anaerobically overnight until stationary.
4. A bacterial suspension was created for each bacterium using a sterile tipped swab to transfer colonies into API suspension media (Biomerieux, Marcy-l'Étoile, France) to reach the turbidity of a 2 MacFarland standard.
5. The starting culture was enumerated.
6. 9.8 mL of the prebiotic media was added into a sterile 15 mL conical tube.
7. 100 μL of each prepared bacterial suspension was added to the media, and it was incubated anaerobically at 37° C. for 48 hours.
8. 100 μL were harvested from each tube containing the bacteria and prebiotic media, it was serially diluted, and plated onto MRS agar and tryptic soy agar (TSA).
9. The bacteria were enumerated on MRS agar (both L. crispatus and S. anginosus). Small colonies were counted as S. anginosus, and large colonies were counted as L. crispatus.
10. S. anginosus was enumerated on TSA plates. Lactobacillus species did not grow well on TSA, whereas S. anginosus did.
11. Background growth of bacteria in the negative control (CHL media without prebiotics) was subtracted from the growth recovered in the presence of the prebiotic mixture.
12. Statistical analysis was performed using Student's t-test. The significance level was set at p<0.05.

### Results

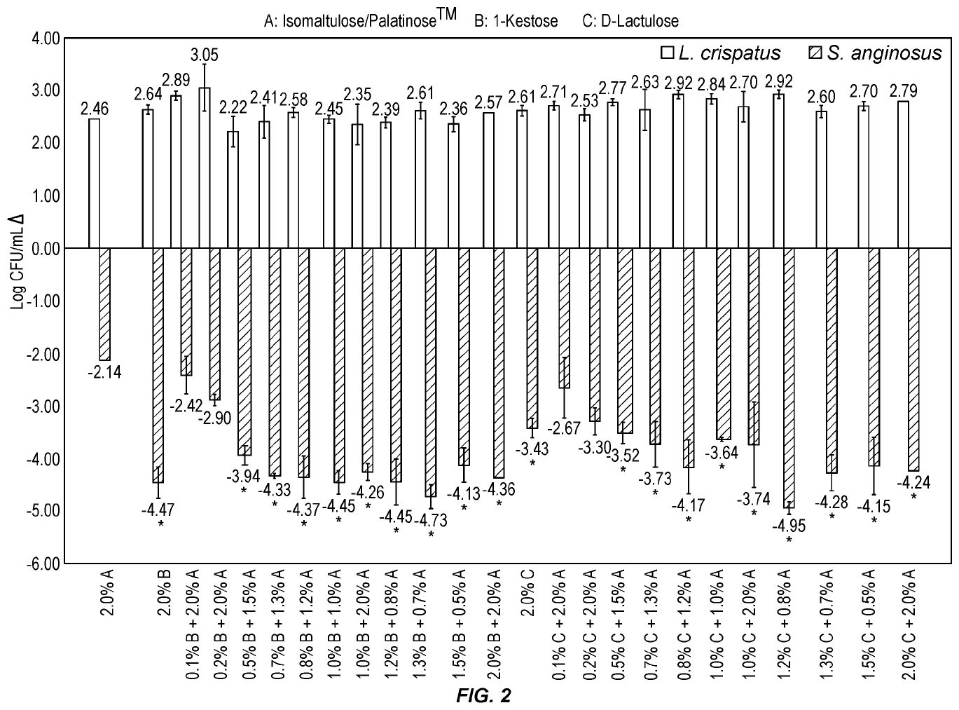

The data generated here is different from previous findings of a single prebiotic compound because a secondary compound was mixed with Palatinose™/isomaltulose. Lactobacillus crispatus recovery for some prebiotic mixtures was on par with previous findings observed with Palatinose™ only (Table 3; FIG. 1). However, S. anginosus recovery was reduced when a secondary compound, such as 1-kestose or lactulose, was added to the mixture (FIG. 2). The compounds 1-kestose and lactulose are not used alone due to their use by other pathogenic bacteria. The decrease in S. anginosus recovery can be due to the rapid metabolizing of 1-kestose (a trisaccharide) and lactulose (a disaccharide) by L. crispatus, leading to lactic acid production release into the external environment. Lactic acid-producing bacteria, such as L. crispatus, have antimicrobial effects on some pathogenic bacterial species.

The data contains unexpected findings which were statistically significant (p<0.05) with the following prebiotic combinations, which displayed the best activity for L. crispatus when tested in competition assays:
Lactulose (1.5% w/v)+Palatinose™ (0.5% w/v) and Lactulose (1.3% w/v)+Palatinose™ (0.7% w/v)
   Palatinose™ (2% w/v) on its own supported a ratio of L. crispatus to S. anginosus of 2,681.
   Lactulose (2% w/v) on its own supported a ratio of L. crispatus to S. anginosus of 37,556.
   However, in the above combinations, the L. crispatus to S. anginosus ratios demonstrated statistical significance (p<0.05) at 932,446 and 572,164, respectively, compared to Palatinose™ (2.0% w/v).
      In addition, the change in the S. anginosus log CFU/mL after the competition assay was statistically significant (p<0.05).
   Two additional L. crispatus to S. anginosus ratios that were statistically significant (<0.05) were Lactulose (1.0% w/v)+Palatinose™ (2.0% w/v) and Lactulose (0.1% w/v)+Palatinose™ (2.0% w/v) at 243,333 and 17,413, respectively.
      However, only the change in S. anginosus log CFU/mL was statistically significant (p<0.05) for Lactulose (1.0% w/v)+Palatinose™ (2.0% w/v).
Combinations of 1-Kestose ranging from 0.1% (w/v) to 1.5% (w/v)+Palatinose™ from 0.5% (w/v) to 2.0% (w/v)·
   Palatinose (2% w/v) on its own supported a ratio of L. crispatus to S. anginosus of 2,681.
   The above combinations were statistically significant (p<0.05), generating L. crispatus to S. anginosus ratios that ranged from 13,005 and 322,274.
      Moreover, most combinations (except 1-Kestose (0.1% w/v)+Palatinose™ (2.0% w/v) and 1-Kestose (0.2% w/v)+Palatinose™ (2.0% w/v)) demonstrated that the S. anginosus log CFU/mL change was statistically significant (p<0.05).
   Although 1-Kestose (2.0% w/v)+Palatinose™ (2.0% w/v) and 1-Kestose (1.0% w/v)+Palatinose™ (2.0% w/v) demonstrated L. crispatus to S. anginosus ratios of 389,223 and 336,363, respectively, the results did not have statistical significance (p>0.05).

In view of these results, the combination of isomaltulose and at least one additional prebiotic (1-kestose or lactulose) results in an unexpected synergistic reaction which is capable of increasing the growth or activity of beneficial microorganisms and decreasing the growth of activity of harmful or unwanted bacteria.

The combination of prebiotics may be used in multiple product forms, including wet wipes and dry wipes, incorporated into liners, pads, and absorbent garments, formulated products including lotions, creams, gels, sprays, and suppositories.

Lastly, while the present disclosure has been provided in detail with respect to certain illustrative and specific aspects thereof, it should not be considered limited to such, as numerous modifications are possible without departing from the broad spirit and scope of the present disclosure as defined in the appended claims.

It will be apparent to those skilled in the art that various modifications and variations can be made in the present disclosure without departing from the scope or spirit thereof. Other implementations of the disclosure will be apparent to those skilled in the art from consideration of the specification and practice of the methods disclosed herein. It is intended that the specification and examples be considered as examples only.

TABLE 1
Tested prebiotic compounds.

| | CAS | | | Catalogue/ |
|---|---|---|---|---|
| Compound | Number | Manufacturer | City, State | Product Number |
| Glucose¹ | 50-99-7 | Sigma-Aldrich | St. Louis, MO | D9434-500G |
| 1-Kestose | 470-69-9 | TCI America | Portland, OR | K0032 |
| D-Lactitol | 81025-04-9 | Sigma-Aldrich | St. Louis, MO | L3520-5G |
| Lactulose | 4618-18-2 | Sigma-Aldrich | St. Louis, MO | L7877-25G |
| Maltitol | 585-88-6 | Sigma-Aldrich | St. Louis, MO | M8892-100G |
| α-Methyl-D-Glucoside | 97-30-3 | Sigma-Aldrich | St. Louis, MO | 66940-100G |
| Palatinose ™/Isomaltulose | 13718-94-0 | Alfa Aesar | Tewksbury, MA | J60091-22 |

¹Positive control for growth and fermentation.

TABLE 2
Prebiotic compound mixtures and their concentrations.

| | Palatinose ™/ | | |
|---|---|---|---|
| | Isomaltulose | Secondary | |
| | Concentration | Prebiotic | Concentration |
| Number | (% w/v) | Compound | (% w/v) |
| 1 | N/A | Glucose¹ | 2.0 |
| 2 | N/A | 1-Kestose | 2.0 |
| 3 | 2.0 | 1-Kestose | 2.0 |
| 4 | 2.0 | 1-Kestose | 1.0 |
| 5 | 2.0 | 1-Kestose | 0.2 |
| 6 | 2.0 | 1-Kestose | 0.1 |
| 7 | 1.5 | 1-Kestose | 0.5 |
| 8 | 1.3 | 1-Kestose | 0.7 |
| 9 | 1.2 | 1-Kestose | 0.8 |
| 10 | 1.0 | 1-Kestose | 1.0 |
| 11 | 0.8 | 1-Kestose | 1.2 |
| 12 | 0.7 | 1-Kestose | 1.3 |
| 13 | 0.5 | 1-Kestose | 1.5 |
| 14 | N/A | D-Lactitol | 2.0 |
| 15 | 2.0 | D-Lactitol | 2.0 |
| 16 | 2.0 | D-Lactitol | 1.0 |
| 17 | 2.0 | D-Lactitol | 0.2 |
| 18 | 2.0 | D-Lactitol | 0.1 |
| 19 | 1.5 | D-Lactitol | 0.5 |
| 20 | 1.3 | D-Lactitol | 0.7 |
| 21 | 1.2 | D-Lactitol | 0.8 |
| 22 | 1.0 | D-Lactitol | 1.0 |
| 23 | 0.8 | D-Lactitol | 1.2 |
| 24 | 0.7 | D-Lactitol | 1.3 |
| 25 | 0.5 | D-Lactitol | 1.5 |
| 26 | N/A | Lactulose | 2.0 |
| 27 | 2.0 | Lactulose | 2.0 |
| 28 | 2.0 | Lactulose | 1.0 |
| 29 | 2.0 | Lactulose | 0.2 |
| 30 | 2.0 | Lactulose | 0.1 |
| 31 | 1.5 | Lactulose | 0.5 |
| 32 | 1.3 | Lactulose | 0.7 |
| 33 | 1.2 | Lactulose | 0.8 |
| 34 | 1.0 | Lactulose | 1.0 |
| 35 | 0.8 | Lactulose | 1.2 |
| 36 | 0.7 | Lactulose | 1.3 |
| 37 | 0.5 | Lactulose | 1.5 |
| 38 | N/A | Maltitol | 2.0 |
| 39 | 1.5 | Maltitol | 0.5 |
| 40 | 1.0 | Maltitol | 1.0 |
| 41 | 0.5 | Maltitol | 1.5 |
| 42 | N/A | α-Methyl-D-Glucoside | 2.0 |
| 43 | 1.5 | α-Methyl-D-Glucoside | 0.5 |
| 44 | 1.0 | α-Methyl-D-Glucoside | 1.0 |
| 45 | 0.5 | α-Methyl-D-Glucoside | 1.5 |
| 46 | 2.0 | N/A | N/A |

¹Glucose was used as the positive control for growth and fermentation in all experiments.

TABLE 3
Comparison of starting inocula, recovered bacteria (log CFU/mL), and ratios of Lactobacillus crispatus KC18-1173-1
to Streptococcus anginosus KC18-1131-3B after exposure to prebiotic compound mixtures in competition assays.

| | | | Log CFU/mL | | | | | Ratio² | |
|---|---|---|---|---|---|---|---|---|---|
| Prebiotic Mixture | | | | Recovered | Δ on | Recovered | Δ on | (L. crispatus/ | P- |
| (% w/v) | Organism | Rep¹ | Start | on MRS | MRS | on TSA | TSA | S. anginosus) | Value³ |
| 1-Kestose (2.0) | S. anginosus | A⁴ | 6.18 | 2.70 | −3.64 | 2.40 | −4.00 | 976,001 | 0.1932 |
| | | B⁵ | 6.60 | 2.30 | −3.63 | 1.70 | −4.00 | | |
| | | C⁶ | 6.60 | 0.00 | −6.61 | 2.74 | −3.74 | | |
| | | D⁷ | 6.40 | 0.00 | −6.08 | 0.00 | −6.45 | | |
| | | E⁸ | 6.77 | 1.70 | −5.03 | 2.54 | −4.15 | | |
| | L. crispatus | A | 4.90 | 7.95 | 2.59 | ND | | | |
| | | B | 4.34 | 8.90 | 3.60 | ND | | | |
| | | C | 5.79 | 8.00 | 2.30 | ND | | | |
| | | D | 5.38 | 7.65 | 1.95 | ND | | | |
| | | E | 5.26 | 8.16 | 2.74 | ND | | | |
| 1-Kestose (0.1) + | S. anginosus | C | 6.60 | 4.00 | −2.61 | 4.48 | −2.00 | 13,005 | 0.0370* |
| Palatinose ™ (2.0) | | E | 6.77 | 0.00 | −6.73 | 3.70 | −3.00 | | |
| | | F⁹ | 6.16 | 3.88 | −2.60 | 4.44 | −2.26 | | |
| | L. crispatus | C | 5.79 | 8.57 | 2.88 | ND | | | |
| | | E | 5.26 | 8.18 | 2.75 | ND | | | |
| | | F | 5.24 | 8.46 | 3.05 | ND | | | |
| 1-Kestose (0.2) + | S. anginosus | C | 6.60 | 4.02 | −2.59 | 4.28 | −2.20 | 44,401 | 0.0170* |
| Palatinose ™ (2.0) | | E | 6.77 | 0.00 | −6.73 | 3.53 | −3.17 | | |
| | | F | 6.16 | 0.00 | −6.48 | 3.38 | −3.32 | | |
| | L. crispatus | C | 5.79 | 8.57 | 2.88 | ND | | | |
| | | E | 5.26 | 8.60 | 3.17 | ND | | | |
| | | F | 5.24 | 8.52 | 3.10 | ND | | | |
| 1-Kestose (0.5) + | S. anginosus | A | 6.18 | 4.63 | −1.71 | 2.60 | −3.80 | 184,473 | 0.0233* |
| Palatinose ™ (1.5) | | F | 6.16 | 0.00 | −6.48 | 2.54 | −4.15 | | |
| | | G¹⁰ | 7.00 | 2.48 | −4.06 | 2.74 | −3.87 | | |
| | L. crispatus | A | 4.90 | 8.10 | 2.74 | ND | | | |
| | | F | 5.24 | 8.00 | 2.59 | ND | | | |
| | | G | 5.48 | 7.13 | 1.32 | ND | | | |
| 1-Kestose (0.7) + | S. anginosus | F | 6.16 | 1.70 | −4.78 | 2.54 | −4.15 | 283,017 | 0.0056* |
| Palatinose ™ (1.3) | | H¹¹ | 6.81 | 0.00 | −6.78 | 2.40 | −4.51 | | |
| | L. crispatus | F | 5.24 | 8.11 | 2.70 | ND | | | |
| | | H | 5.13 | 7.60 | 2.12 | ND | | | |
| 1-Kestose (0.8) + | S. anginosus | H | 6.81 | 0.00 | −6.78 | 2.54 | −4.36 | 322,274 | 0.0015* |
| Palatinose ™ (1.2) | | I¹² | 5.88 | 0.00 | −6.70 | 2.70 | −4.30 | | |
| | | J¹³ | 5.85 | 0.00 | −6.54 | 2.31 | −4.44 | | |
| | L. crispatus | H | 5.13 | 8.16 | 2.68 | ND | | | |
| | | I | 5.48 | 7.98 | 3.05 | ND | | | |
| | | J | 5.32 | 8.00 | 2.00 | ND | | | |
| 1-Kestose (1.0) + | S. anginosus | A | 6.18 | 0.00 | −6.34 | 1.70 | −4.70 | 291,870 | 0.0220* |
| Palatinose ™ (1.0) | | F | 6.16 | 0.00 | −6.48 | 1.70 | −5.00 | | |
| | | G | 7.00 | 2.85 | −3.69 | 2.95 | −3.66 | | |
| | L. crispatus | A | 4.90 | 7.95 | 2.59 | ND | | | |
| | | F | 5.24 | 7.90 | 2.49 | ND | | | |
| | | G | 5.48 | 8.08 | 2.27 | ND | | | |
| 1-Kestose (1.0) + | S. anginosus | C | 6.60 | 0.00 | −6.61 | 2.00 | −4.48 | 336,363 | 0.0776 |
| Palatinose ™ (2.0) | | E | 6.77 | 2.70 | −4.03 | 2.65 | −4.05 | | |
| | L. crispatus | C | 5.79 | 8.13 | 2.43 | ND | | | |
| | | E | 5.26 | 7.70 | 2.28 | ND | | | |
| 1-Kestose (1.2) + | S. anginosus | I | 5.88 | 0.00 | −6.70 | 2.58 | −4.42 | 234,513 | 0.0130* |
| Palatinose ™ (0.8) | | J | 5.85 | 1.70 | −4.85 | 2.00 | −4.75 | | |
| | | K¹⁴ | 6.93 | 0.00 | −7.00 | 2.81 | −4.19 | | |
| | L. crispatus | I | 5.48 | 8.02 | 3.10 | ND | | | |
| | | J | 5.32 | 7.78 | 1.78 | ND | | | |
| | | K | 5.30 | 8.00 | 2.30 | ND | | | |
| 1-Kestose (1.3) + | S. anginosus | F | 6.16 | 0.00 | −6.48 | 1.70 | −5.00 | 217,391 | 0.0228* |
| Palatinose ™ (0.7) | | H | 6.81 | 3.30 | −3.48 | 3.02 | −3.88 | | |
| | | I | 5.88 | 0.00 | −6.70 | 1.70 | −5.30 | | |
| | L. crispatus | F | 5.24 | 7.85 | 2.43 | ND | | | |
| | | H | 5.13 | 8.11 | 2.63 | ND | | | |
| | | I | 5.48 | 7.70 | 2.78 | ND | | | |
| 1-Kestose (1.5) + | S. anginosus | A | 6.18 | 0.00 | −6.34 | 2.00 | −4.40 | 182,504 | 0.0431* |
| Palatinose ™ (0.5) | | G | 7.00 | 2.48 | −4.06 | 2.93 | −3.68 | | |
| | | H | 6.81 | 0.00 | −6.78 | 2.60 | −4.30 | | |
| | L. crispatus | A | 4.90 | 7.85 | 2.49 | ND | | | |
| | | G | 5.48 | 7.85 | 2.04 | ND | | | |
| | | H | 5.13 | 8.02 | 2.54 | ND | | | |
| 1-Kestose (2.0) + | S. anginosus | C | 6.60 | 2.30 | −4.31 | 2.48 | −4.00 | 389,223 | 0.1402 |
| Palatinose ™ (2.0) | | E | 6.77 | 0.00 | −6.73 | 1.70 | −5.00 | | |
| | | H | 6.81 | 4.30 | −2.48 | 2.81 | −4.09 | | |
| | L. crispatus | C | 5.79 | 8.00 | 2.30 | ND | | | |
| | | E | 5.26 | 8.23 | 2.81 | ND | | | |
| | | H | 5.13 | 8.08 | 2.59 | ND | | | |
| D-Lactitol (2.0) | S. anginosus | L¹⁵ | 6.15 | 7.45 | 0.75 | 7.43 | 0.58 | 0 | 0.1610 |
| | | M¹⁶ | 6.90 | 7.54 | 0.69 | 7.65 | 0.52 | | |
| | L. crispatus | L | 5.22 | 5.90 | 0.77 | ND | | | |
| | | M | 5.10 | 6.74 | 0.66 | ND | | | |
| D-Lactitol (0.5) + | S. anginosus | A | 6.18 | 6.08 | −0.26 | 5.98 | −0.42 | 42 | 0.1640 |
| Palatinose ™ (1.5) | | N¹⁷ | 6.74 | 5.72 | −0.70 | 5.86 | −0.62 | | |
| | L. crispatus | A | 4.90 | 7.81 | 2.45 | ND | | | |
| | | N | 5.32 | 6.88 | 1.00 | ND | | | |
| D-Lactitol (1.0) + | S. anginosus | A | 6.18 | 6.48 | 0.14 | 6.70 | 0.30 | 1 | 0.1611 |
| Palatinose ™ (1.0) | | N | 6.74 | 5.92 | −0.50 | 5.80 | −0.68 | | |
| | L. crispatus | A | 4.90 | 6.90 | 1.54 | ND | | | |
| | | N | 5.32 | 6.26 | 0.38 | ND | | | |
| D-Lactitol (1.5) + | S. anginosus | A | 6.18 | 6.60 | 0.26 | 6.81 | 0.41 | 0 | 0.1610 |
| Palatinose ™ (0.5) | | N | 6.74 | 6.10 | −0.32 | 6.02 | −0.46 | | |
| | L. crispatus | A | 4.90 | 6.48 | 1.12 | ND | | | |
| | | N | 5.32 | 5.72 | −0.16 | ND | | | |
| Lactulose (2.0) | S. anginosus | A | 6.18 | 4.11 | −2.23 | 4.23 | −2.17 | 37,556 | 0.1338 |
| | | B | 6.60 | 3.40 | −2.53 | 2.00 | −3.70 | | |
| | | C | 6.60 | 0.00 | −6.61 | 2.74 | −3.74 | | |
| | | D | 6.40 | 0.00 | −6.08 | 2.78 | −3.67 | | |
| | | E | 6.77 | 0.00 | −6.73 | 2.85 | −3.85 | | |
| | L. crispatus | A | 4.90 | 7.74 | 2.38 | ND | | | |
| | | B | 4.34 | 8.10 | 2.80 | ND | | | |
| | | C | 5.79 | 8.28 | 2.58 | ND | | | |
| | | D | 5.38 | 8.30 | 2.60 | ND | | | |
| | | E | 5.26 | 8.11 | 2.69 | ND | | | |
| Lactulose (0.1) + | S. anginosus | C | 6.60 | 3.74 | −2.87 | 4.13 | −2.35 | 17,413 | 0.0043* |
| Palatinose ™ (2.0) | | E | 6.77 | 4.00 | −2.73 | 3.74 | −2.96 | | |
| | | F | 6.16 | 4.13 | −2.35 | 4.00 | −2.70 | | |
| | L. crispatus | C | 5.79 | 8.23 | 2.53 | ND | | | |
| | | E | 5.26 | 8.13 | 2.71 | ND | | | |
| | | F | 5.24 | 8.30 | 2.89 | ND | | | |
| Lactulose (0.2) + | S. anginosus | C | 6.60 | 3.88 | −2.73 | 4.00 | −2.48 | 36,477 | 0.2024 |
| Palatinose ™ (2.0) | | E | 6.77 | 3.54 | −3.18 | 3.70 | −3.00 | | |
| | | J | 5.85 | 0.00 | −6.54 | 2.33 | −4.42 | | |
| | L. crispatus | C | 5.79 | 8.33 | 2.63 | ND | | | |
| | | E | 5.26 | 8.00 | 2.58 | ND | | | |
| | | J | 5.32 | 8.38 | 2.38 | ND | | | |
| Lactulose (0.5) + | S. anginosus | A | 6.18 | 3.40 | 2.94 | 3.24 | −3.16 | 120,034 | 0.1510 |
| Palatinose ™ (1.5) | | B | 6.60 | 3.74 | −2.19 | 1.70 | −4.00 | | |
| | | F | 6.16 | 3.30 | −3.18 | 3.30 | −3.40 | | |
| | L. crispatus | A | 4.90 | 8.11 | 2.75 | ND | | | |
| | | B | 4.34 | 7.88 | 2.58 | ND | | | |
| | | F | 5.24 | 8.40 | 2.98 | ND | | | |
| Lactulose (0.7) + | S. anginosus | F | 6.16 | 0.00 | 6.48 | 3.34 | −3.36 | 160,000 | 0.1216 |
| Palatinose ™ (1.3) | | H | 6.81 | 2.65 | −4.12 | 3.11 | −3.79 | | |
| | | J | 5.85 | 0.00 | −6.54 | 2.70 | −4.05 | | |
| | L. crispatus | F | 5.24 | 8.18 | 2.76 | ND | | | |
| | | H | 5.13 | 8.04 | 2.56 | ND | | | |
| | | J | 5.32 | 8.58 | 2.58 | ND | | | |
| Lactulose (0.8) + | S. anginosus | I | 6.18 | 0.00 | −7.00 | 2.40 | −4.90 | 223,636 | 0.0850 |
| Palatinose ™ (1.2) | | F | 6.16 | 0.00 | −6.48 | 3.30 | −3.40 | | |
| | | H | 6.81 | 0.00 | −6.78 | 2.70 | −4.20 | | |
| | L. crispatus | F | 5.24 | 7.70 | 2.28 | ND | | | |
| | | H | 5.13 | 8.33 | 2.85 | ND | | | |
| | | I | 5.48 | 8.54 | 3.62 | ND | | | |
| Lactulose (1.0) + | S. anginosus | A | 6.18 | 3.74 | −2.60 | 2.74 | −3.66 | 320,142 | 0.0969 |
| Palatinose ™ (1.0) | | B | 6.60 | 3.18 | −2.75 | 2.95 | −2.75 | | |
| | | F | 6.16 | 0.00 | −6.48 | 2.18 | −4.52 | | |
| | L. crispatus | A | 4.90 | 8.24 | 2.88 | ND | | | |
| | | B | 4.34 | 7.98 | 2.68 | ND | | | |
| | | F | 5.24 | 8.38 | 2.97 | ND | | | |
| Lactulose (1.0) + | S. anginosus | C | 6.60 | 0.00 | −6.61 | 2.70 | −3.78 | 243,333 | 0.0066* |
| Palatinose ™ (2.0) | | E | 6.77 | 2.70 | −4.03 | 3.00 | −3.70 | | |
| | L. crispatus | C | 5.79 | 8.30 | 2.60 | ND | | | |
| | | E | 5.26 | 8.22 | 2.79 | ND | | | |
| Lactulose (1.2) + | S. anginosus | F | 6.16 | 0.00 | −6.48 | 2.18 | | 465,382 | 0.2886 |
| Palatinose ™ (0.8) | | H | 6.81 | 0.00 | −6.78 | 3.24 | | | |
| | | I | 6.18 | 0.00 | −7.00 | 0.00 | | | |
| | | J | 5.85 | 0.00 | −6.54 | 2.45 | | | |
| | L. crispatus | F | 5.24 | 8.40 | 2.98 | ND | | | |
| | | H | 5.13 | 8.16 | 2.68 | ND | | | |
| | | I | 5.48 | 8.60 | 3.67 | ND | | | |
| | | J | 5.32 | 8.35 | 2.35 | ND | | | |
| Lactulose (1.3) + | S. anginosus | F | 6.16 | 0.00 | −6.48 | 2.60 | −4.10 | 572,164 | 0.0030* |
| Palatinose ™ (0.7) | | H | 6.81 | 0.00 | −6.78 | 2.40 | −4.51 | | |
| | | J | 5.85 | 0.00 | −6.54 | 2.51 | −4.25 | | |
| | L. crispatus | F | 5.24 | 8.18 | 2.76 | ND | | | |
| | | H | 5.13 | 8.06 | 2.58 | ND | | | |
| | | J | 5.32 | 8.46 | 2.46 | ND | | | |
| Lactulose (1.5) + | S. anginosus | A | 6.18 | 0.00 | −6.34 | 2.18 | −4.22 | 932,446 | 0.0238* |
| Palatinose ™ (0.5) | | B | 6.60 | 3.18 | −2.75 | 2.18 | −3.52 | | |
| | | F | 6.16 | 1.70 | −4.78 | 2.00 | −4.70 | | |
| | L. crispatus | A | 4.90 | 7.85 | 2.49 | ND | | | |
| | | B | 4.34 | 8.02 | 2.72 | ND | | | |
| | | F | 5.24 | 8.30 | 2.89 | ND | | | |
| Lactulose (2.0) + | S. anginosus | C | 6.60 | 0.00 | −6.61 | 1.70 | −4.78 | 428,571 | 0.0964 |
| Palatinose ™ (2.0) | | E | 6.77 | 2.74 | −3.99 | 3.00 | −3.70 | | |
| | L. crispatus | C | 5.79 | 8.40 | 2.70 | ND | | | |
| | | E | 5.26 | 8.30 | 2.88 | ND | | | |
| Maltitol (2.0) | S. anginosus | L | 6.15 | 6.58 | −0.12 | 7.26 | 0.41 | 0 | 0.1610 |
| | | M | 6.90 | 6.48 | −0.37 | 7.60 | 0.47 | | |
| | L. crispatus | L | 5.22 | 5.48 | 0.35 | ND | | | |
| | | M | 5.10 | 6.00 | −0.08 | ND | | | |
| Maltitol (0.5) + | S. anginosus | A | 6.18 | 5.65 | −0.69 | 5.70 | −0.70 | 21 | 0.1626 |
| Palatinose ™ (1.5) | | N | 6.74 | 5.55 | −0.87 | 5.60 | −0.88 | | |
| | L. crispatus | A | 4.90 | 7.06 | 1.70 | ND | | | |
| | | N | 5.32 | 6.90 | 1.02 | ND | | | |
| Maltitol (1.0) + | S. anginosus | A | 6.18 | 6.65 | 0.31 | 6.60 | 0.20 | 0 | 0.1610 |
| Palatinose ™ (1.0) | | N | 6.74 | 5.68 | −0.74 | 5.90 | −0.58 | | |
| | L. crispatus | A | 4.90 | 6.00 | 0.64 | ND | | | |
| | | N | 5.32 | 5.12 | −0.76 | ND | | | |
| Maltitol (1.5) + | S. anginosus | A | 6.18 | 6.60 | 0.26 | 6.40 | 0.00 | 0 | 0.1610 |
| Palatinose ™ (0.5) | | N | 6.74 | 6.32 | −0.10 | 6.15 | −0.33 | | |
| | L. crispatus | A | 4.90 | 5.18 | −0.18 | ND | | | |
| | | N | 5.32 | 4.64 | −1.24 | ND | | | |
| α-Methyl-D-Glucoside (2.0) | S. anginosus | B | 6.60 | 5.60 | −0.33 | 6.04 | 0.34 | 0 | 0.0848 |
| | | L | 6.15 | 7.15 | 0.45 | 7.47 | 0.62 | | |
| | | M | 6.90 | 7.40 | 0.55 | 7.54 | 0.41 | | |
| | L. crispatus | B | 4.34 | 7.20 | 1.90 | ND | | | |
| | | L | 5.22 | 6.52 | 1.39 | ND | | | |
| | | M | 5.10 | 6.78 | 0.70 | ND | | | |
| α-Methyl-D-Glucoside (0.5) + | S. anginosus | A | 6.18 | 5.48 | −0.86 | 6.00 | −0.40 | 32 | 0.1633 |
| Palatinose ™ (1.5) | | N | 6.74 | 5.32 | −1.10 | 5.80 | −0.68 | | |
| | L. crispatus | A | 4.90 | 7.60 | 2.24 | ND | | | |
| | | N | 5.32 | 7.12 | 1.24 | ND | | | |
| α-Methyl-D-Glucoside (1.0) + | S. anginosus | A | 6.18 | 5.70 | −0.64 | 5.70 | −0.70 | 121 | 0.1697 |
| Palatinose ™ (1.0) | | N | 6.74 | 5.50 | −0.92 | 5.52 | −0.96 | | |
| | L. crispatus | A | 4.90 | 7.93 | 2.57 | ND | | | |
| | | N | 5.32 | 7.20 | 1.32 | ND | | | |
| α-Methyl-D-Glucoside (1.5) + | S. anginosus | A | 6.18 | 5.40 | −0.94 | 5.70 | −0.70 | 151 | 0.1745 |
| Palatinose ™ (0.5) | | N | 6.74 | 5.24 | −1.18 | 5.10 | −1.38 | | |
| | L. crispatus | A | 4.90 | 7.85 | 2.49 | ND | | | |
| | | N | 5.32 | 7.38 | 1.50 | ND | | | |
| Palatinose ™ (2.0) | S. anginosus | C | 6.60 | 4.78 | −1.83 | 5.00 | −1.48 | 2,681 | NA |
| | | D | 6.40 | 4.00 | −2.08 | 4.04 | −2.41 | | |
| | | E | 6.77 | 4.70 | −2.03 | 4.70 | −2.00 | | |
| | | L | 6.15 | 4.88 | −1.82 | 4.53 | −2.42 | | |
| | | M | 6.90 | 5.16 | −1.69 | 4.74 | 2.39 | | |

| | | | | | |
|---|---|---|---|---|---|
| L. crispatus | C | 5.79 | 7.93 | 2.10 | ND |
| | D | 5.38 | 8.00 | 2.30 | ND |
| | E | 5.26 | 8.19 | 2.77 | ND |
| | L | 5.22 | 8.37 | 3.24 | ND |
| | M | 5.10 | 7.98 | 1.90 | ND |

ND, organism not detected;
NA, not applicable.
¹The log CFU/mL background growth in API ® 50 CHL media without an added prebiotic compound was subtracted from the growth of prebiotic mixtures for each organism. Each replicate has a footnote with the subtracted log CFU/mL values.
²The ratio was determined by dividing the average CFU/mL recovered for L. crispatus KC18-1173-1 on MRS media by the average CFU/mL recovered for S. anginosus KC18-1131-3B on TSA.
³Student's t-test was performed to determine if the ratio differences of individual prebiotic combinations were statistically significant compared to Palatinose ™/isomaltulose (2% w/v). The significance level was set at p < 0.05, indicated by (*).
⁴Replicate A background growth for S. anginosus KC18-1131-3B was 0.16 log CFU/mL on MRS and 0.22 log CFU/mL on TSA, and 0.46 log CFU/mL for L. crispatus KC18-1173-1 on MRS.
⁵Replicate B background growth for S. anginosus KC18-1131-3B was −0.67 log CFU/mL on MRS and −0.90 log CFU/mL on TSA, and 0.96 log CFU/mL for L. crispatus KC18-1173-1 on MRS.
⁶Replicate C background growth for S. anginosus KC18-1131-3B was 0.01 log CFU/mL on MRS and −0.12 log CFU/mL on TSA, and −0.09 log CFU/mL for L. crispatus KC18-1173-1 on MRS.
⁷Replicate D background growth for S. anginosus KC18-1131-3B was −0.32 log CFU/mL on MRS and 0.05 log CFU/mL on TSA, and 0.32 log CFU/mL for L. crispatus KC18-1173-1 on MRS.
⁸Replicate E background growth for S. anginosus KC18-1131-3B was −0.04 log CFU/mL on MRS and −0.07 log CFU/mL on TSA, and 0.17 log CFU/mL for L. crispatus KC18-1173-1 on MRS.
⁹Replicate F background growth for S. anginosus KC18-1131-3B was 0.32 log CFU/mL on MRS and 0.54 log CFU/mL on TSA, and 0.17 log CFU/mL for L. crispatus KC18-1173-1 on MRS.
¹⁰Replicate G background growth for S. anginosus KC18-1131-3B was −0.46 log CFU/mL on MRS and −0.39 log CFU/mL on TSA, and 0.33 log CFU/mL for L. crispatus KC18-1173-1 on MRS.
¹¹Replicate H background growth for S. anginosus KC18-1131-3B was −0.03 log CFU/mL on MRS and 0.09 log CFU/mL on TSA, and 0.35 log CFU/mL for L. crispatus KC18-1173-1 on MRS.
¹²Replicate I background growth for S. anginosus KC18-1131-3B was 0.83 log CFU/mL on MRS and 1.12 log CFU/mL on TSA, and −0.56 log CFU/mL for L. crispatus KC18-1173-1 on MRS.
¹³Replicate J background growth for S. anginosus KC18-1131-3B was 0.70 log CFU/mL on MRS and 0.91 log CFU/mL on TSA, and 0.68 log CFU/mL for L. crispatus KC18-1173-1 on MRS.
¹⁴Replicate K background growth for S. anginosus KC18-1131-3B was 0.07 log CFU/mL on MRS and 0.07 log CFU/mL on TSA, and 0.40 log CFU/mL for L. crispatus KC18-1173-1 on MRS.
¹⁵Replicate L background growth for S. anginosus KC18-1131-3B was 0.55 log CFU/mL on MRS and 0.70 log CFU/mL on TSA, and −0.09 log CFU/mL for L. crispatus KC18-1173-1 on MRS.
¹⁶Replicate M background growth for S. anginosus KC18-1131-3B was −0.05 log CFU/mL on MRS and 0.23 log CFU/mL on TSA, and 0.98 log CFU/mL for L. crispatus KC18-1173-1 on MRS.
¹⁷Replicate N background growth for S. anginosus KC18-1131-3B was −0.32 log CFU/mL on MRS and −0.26 log CFU/mL on TSA, and 0.56 log CFU/mL for L. crispatus KC18-1173-1 on MRS.

### REFERENCES

1. Li, J., Peed, L. A., Joyner, C. F., Vongsa, R. A., Koenig, D. W., & Bartell, R. D. (2015). Composition for maintaining Lactobacillus dominance. United States Patent Publication No. US 2018/0256615 A1.
2. Zeng, Z., Zhang W., & Wu, K. Shenzhen (2019). Composition for vagina and antimicrobial agent combination for use in preparing composition for vagina. United States Patent Publication No. US 2019/0231748 A1.
3. Zeng, Z., & Zhou, R. (2016). Vaginal composition and use thereof. United States Patent Publication No. US 2016/0375045 A1.
4. Vongsa, R. A., Minerath, R. A., Busch, M. A., Tan, J., & Koenig, D. W. (2016). In vitro evaluation of nutrients that selectively confer a competitive advantage to lactobacilli. Beneficial Microbes, 7, 299-304.
5. Akgül T, Karakan T. The role of probiotics in women with recurrent urinary tract infections. Turk J Urol. 2018 September; 44 (5): 377-383. doi: 10.5152/tud.2018.48742. Epub 2018 Sep. 1. PMID: 30487041; PMCID: PMC6134985.
6. Weysser Felipe Cândido de Souza, Francisco Lucas Chaves Almeida, Ruann Janser Soares de Castro, Hélia Harumi Sato, Isomaltulose: From origin to application and its beneficial properties—A bibliometric approach, Food Research International, Volume 155, 2022.
7. Barea-Alvarez M, Benito M T, Olano A, Jimeno M L, Moreno F J. Synthesis and characterization of isomaltulose-derived oligosaccharides produced by transglucosylation reaction of Leuconostoc mesenteroides dextransucrase. J Agric Food Chem. 2014 Sep. 17; 62 (37): 9137-44.
8. Kashimura J, Nagai Y, Goda T. Inhibitory action of palatinose and its hydrogenated derivatives on the hydrolysis of alpha-glucosylsaccharides in the small intestine. J Agric Food Chem. 2008 Jul. 23; 56 (14): 5892-5.
9. Li T, Liu Z, Zhang X, Chen X, Wang S. Local Probiotic Lactobacillus crispatus and Lactobacillus delbrueckii Exhibit Strong Antifungal Effects Against Vulvovaginal Candidiasis in a Rat Model. Front Microbiol. 2019 May 8; 10:1033.
10. Verhelst R, Verstraelen H, Claeys G, Verschraegen G, Van Simaey L, De Ganck C, De Backer E, Temmerman M, Vaneechoutte M. Comparison between Gram stain and culture for the characterization of vaginal microflora: definition of a distinct grade that resembles grade I microflora and revised categorization of grade I microflora. BMC Microbiol. 2005 Oct. 14; 5:61.
11. Wu L, Wu S, Qiu J, Xu C, Li S, Xu H. Green synthesis of isomaltulose from cane molasses by Bacillus subtilis WB800-pHA01-palI in a biologic membrane reactor.

### Food Chem. 2017 Aug. 15; 229:761-768.

12. Chee, W. J. Y., Chew, S. Y. & Than, L. T. L. Vaginal microbiota and the potential of Lactobacillus derivatives in maintaining vaginal health. Microb Cell Fact 19, 203 (2020).

### Embodiments

Embodiment 1: A prebiotic formulation comprising prebiotic agents isomaltulose and 1-kestose.

Embodiment 2: The prebiotic formulation of embodiment 1, wherein the prebiotic formulation further comprises at least one additional prebiotic agent.

Embodiment 3: The prebiotic formulation of embodiment 3, wherein said additional prebiotic agent is lactulose.

Embodiment 4: The prebiotic formulation of any one of embodiments 1-3, wherein the prebiotic formulation further comprises a carrier.

Embodiment 5: The prebiotic formulation of any one of embodiments 1-4, wherein the prebiotic agents comprise 0.01% to 20% w/v of the prebiotic formulation.

Embodiment 6: The prebiotic formulation of embodiment 5, wherein the prebiotic agents comprise 1.0% to 3.0% w/v.

Embodiment 7: The prebiotic formulation of embodiment 6, wherein the prebiotic agents comprise 2.0% w/v.

Embodiment 8: The prebiotic formulation of embodiment 7, wherein the isomaltulose is present in the prebiotic formulation at a concentration of 1.2% w/v, and the 1-kestose is present at 0.8% w/v.

Embodiment 9: The prebiotic formulation of embodiment 7, wherein the isomaltulose is present in the prebiotic formulation at a concentration of 1.3% w/v, and the 1-kestose is present at 0.7% w/v.

Embodiment 10: The prebiotic formulation of embodiment 7, wherein the isomaltulose is present in the prebiotic formulation at a concentration of 0.8% w/v, and the 1-kestose is present at 1.2% w/v.

Embodiment 11: The prebiotic formulation of embodiment 7, wherein the isomaltulose is present in the prebiotic formulation at a concentration of 0.7% w/v, and the 1-kestose is present at 1.3% w/v.

Embodiment 12: The prebiotic formulation of embodiment 6, wherein the isomaltulose is present in the prebiotic formulation at a concentration of 2.0% w/v, and the 1-kestose is present at 0.2% w/V.

Embodiment 13: The prebiotic formulation of embodiment 6, wherein the isomaltulose is present in the prebiotic formulation at a concentration of 2.0% w/v, and the 1-kestose is present at 0.1% w/v.

Embodiment 14: The prebiotic formulation of embodiment 7, wherein the isomaltulose is present in the prebiotic formulation at a concentration of 1.5% w/v, and the 1-kestose is present at 0.5% w/v.

Embodiment 15: The prebiotic formulation of embodiment 7, wherein the isomaltulose is present in the prebiotic formulation at a concentration of 1.0% w/v, and the 1-kestose is present at 1.0% w/v.

Embodiment 16: The prebiotic formulation of embodiment 7, wherein the isomaltulose is present in the prebiotic formulation at a concentration of 0.5% w/v, and the 1-kestose is present at 1.5% w/v.

Embodiment 17: A prebiotic formulation comprising prebiotic agents isomaltulose and lactulose.

Embodiment 18: The prebiotic formulation of embodiment 11, wherein the prebiotic formulation further comprises at least one additional prebiotic agent.

Embodiment 19: The prebiotic formulation of embodiment 12, wherein said additional prebiotic agent is 1-kestose.

Embodiment 20: The prebiotic formulation of any one of embodiments 11-13, wherein the prebiotic formulation further comprises a carrier.

Embodiment 21: The prebiotic formulation of any one of embodiments 11-14, wherein the prebiotic agents comprise 0.01% to 20% w/v of the prebiotic formulation.

Embodiment 22: The prebiotic formulation of embodiment 15, wherein the prebiotic agents comprise 1.0% to 3.0% w/v.

Embodiment 23: The prebiotic formulation of embodiment 16, wherein the prebiotic agents comprise 2.0% w/v.

Embodiment 24: The prebiotic formulation of embodiment 17, wherein the isomaltulose is present in the prebiotic formulation at a concentration of 1.5% w/v, and the lactulose is present at 0.5% w/v.

Embodiment 25: The prebiotic formulation of embodiment 23, wherein the isomaltulose is present in the prebiotic formulation at a concentration of 0.7% w/v, and the lactulose is present at 1.3% w/v.

Embodiment 26: The prebiotic formulation of embodiment 23, wherein the isomaltulose is present in the prebiotic formulation at a concentration of 1.0% w/v, and the lactulose is present at 1.0% w/v.

Embodiment 27: The prebiotic formulation of embodiment 23, wherein the isomaltulose is present in the prebiotic formulation at a concentration of 0.5% w/v, and the lactulose is present at 1.5% w/v.

Embodiment 28: The prebiotic formulation of embodiment 22, wherein the isomaltulose is present in the prebiotic formulation at a concentration of 2.0% w/v, and the lactulose is present at 1.0% w/v.

Embodiment 29: The prebiotic formulation of embodiment 22, wherein the isomaltulose is present in the prebiotic formulation at a concentration of 2.0% w/v, and the lactulose is present at 0.1% w/v.

Embodiment 21: A method of modulating growth of urogenital microorganisms in a subject, the method comprising administering to the subject a prebiotic formulation, wherein the prebiotic formulation comprises prebiotic agents, wherein the prebiotic agents comprise isomaltulose and at least one of 1-kestose and lactulose.

Embodiment 22: The method of embodiment 21, wherein the prebiotic formulation further comprises a carrier.

Embodiment 23: The method of embodiment 22, wherein the carrier is a cream, liquid, solution, spray, paste or gel.

Embodiment 24: The method of embodiment 23, wherein the carrier is water.

Embodiment 25: The method of any one of embodiments 21-24, wherein the prebiotic formulation increases the growth of at least one beneficial bacteria.

Embodiment 26: The method of embodiment 25, wherein the beneficial bacteria is Lactobacillus.

Embodiment 27: The method of embodiment 26, wherein the beneficial bacteria is Lactobacillus crispatus.

Embodiment 28: The method of any one of embodiments 21-25, wherein the prebiotic formulation decreases the growth of at least one pathogenic bacteria.

Embodiment 29: The method of embodiment 28, wherein the pathogenic bacteria is Streptococcus anginosus.

Embodiment 30: The method of any one of embodiments 21-29, wherein the ratio of isomaltulose to at least one of 1-kestose and lactulose is 5:1, 4:1, 3:1, 2:1, 1:1, 1:2, 1:3, 1:4, or 1:5.

Embodiment 31: The method of any one of embodiments 21-30, wherein the prebiotic formulation is given topically.

Embodiment 32: The method of embodiment 31, wherein the prebiotic formulation has been applied to a substrate.

Embodiment 33: The method of embodiment 32, wherein the substrate is an absorbent material.

Embodiment 34: The method of embodiment 33, wherein the absorbent material is a wipe, sheet, pad, or diaper.

Embodiment 35: A method of treating or preventing urogenital dysbiosis, the method comprising administering to the subject a prebiotic formulation, wherein the prebiotic formulation comprises prebiotic agents, wherein the prebiotic agents comprise isomaltulose and at least one of 1-kestose and lactulose.

Embodiment 36: The method of embodiment 35, wherein the subject has, or is at risk of developing, an overactive bladder, urinary urge incontinence, or urinary tract infection.

Embodiment 37: The method of embodiment 36, wherein the subject has been diagnosed as having an overactive bladder, urinary urge incontinence, or urinary tract infection.

Embodiment 38: The method of embodiment 36, wherein the subject has been diagnosed as being at risk of developing an overactive bladder, urinary urge incontinence, or urinary tract infection.

Embodiment 39: The method of any one of embodiments 35-38, wherein the prebiotic formulation further comprises a carrier.

Embodiment 40: The method of embodiment 39, wherein the carrier is a cream, liquid, solution, spray, paste or gel.

Embodiment 41: The method of embodiment 40, wherein the carrier is water.

Embodiment 42: The method of any one of embodiments 35-41, wherein the ratio of isomaltulose to at least one of 1-kestose and lactulose.

Embodiment 43: The method of any one of embodiments 35-42, wherein the prebiotic formulation is given topically.

Embodiment 44: The method of embodiment 43, wherein the prebiotic formulation has been applied to a substrate.

Embodiment 45: The method of embodiment 44, wherein the substrate is an absorbent material.

Embodiment 46: The method of embodiment 45, wherein the absorbent material is a wipe, sheet, pad, or diaper.

What is claimed is:

1. A prebiotic formulation comprising prebiotic agents isomaltulose and 1-kestose.

2. The prebiotic formulation of claim 1, wherein the prebiotic formulation further comprises at least one additional prebiotic agent.

3. The prebiotic formulation of claim 2, wherein said additional prebiotic agent comprises lactulose.

4. The prebiotic formulation of claim 1, wherein the prebiotic formulation further comprises a carrier.

5. The prebiotic formulation of claim 1, wherein the prebiotic agents comprise 0.01% to 20% w/v of the prebiotic formulation.

6. A prebiotic formulation comprising prebiotic agents isomaltulose and lactulose.

7. The prebiotic formulation of claim 6, wherein the prebiotic formulation further comprises at least one additional prebiotic agent.

8. The prebiotic formulation of claim 7, wherein said additional prebiotic agent comprises 1-kestose.

9. The prebiotic formulation of claim 6, wherein the prebiotic formulation further comprises a carrier.

10. The prebiotic formulation of claim 6, wherein the prebiotic agents comprise 0.01% to 20% w/v of the prebiotic formulation.

11. A method of modulating growth of urogenital microorganisms in a subject, the method comprising administering to the subject a prebiotic formulation, wherein the prebiotic formulation comprises prebiotic agents, wherein the prebiotic agents comprise isomaltulose and at least one of 1-kestose and lactulose.

12. The method of claim 11, wherein the prebiotic formulation further comprises a carrier, wherein the carrier is a cream, liquid, solution, soap, spray, paste, or gel.

13. The method of claim 11, wherein the prebiotic formulation increases the growth of at least one beneficial bacterium.

14. The method of claim 13, wherein the beneficial bacteria is Lactobacillus crispatus.

15. The method of any claim 11, wherein the prebiotic formulation decreases the growth of at least one pathogenic bacterium.

16. The method of claim 15, wherein the pathogenic bacteria is Streptococcus anginosus.

17. The method of claim 11, wherein isomaltulose and at least one of 1-kestose and/or lactulose are present at a ratio of 20:1, 13:7, 10:1, 5:1, 4:1, 3:1, 3:2, 2:1, 1:1, 1:2, 2:3, 1:3, 1:4, 1:5, or 7:13.

18. The method of claim 11, wherein the prebiotic formulation is given topically.

19. The method of claim 11, wherein the prebiotic formulation has been applied to a substrate, wherein the substrate is an absorbent material.

20. The method of claim 19, wherein the absorbent material is a wipe, sheet, pad, or diaper.
